(19) United States
(12) Patent Application Publication
(10) Pub. No.: US 2012/0199119 A1
(43) Pub. Date: Aug. 9, 2012

(54) DISTRIBUTION HEAD FOR A DEVICE FOR DISTRIBUTING A FLUID PRODUCT

(75) Inventor: Jean-Marc Pardonge, Les Authieux Sur Port St Ouen (FR)

(73) Assignee: VALOIS SAS, Le Neubourg (FR)

(21) Appl. No.: 13/501,593

(22) PCT Filed: Oct. 20, 2010
(86) PCT No.: PCT/FR10/52237

§ 371 Date: Apr. 12, 2012

(30) Foreign Application Priority Data
Oct. 20, 2009 (FR) 09 57327

(51) Int. Cl. A61M 11/00; B05B 1/00; A61J 1/14

(52) U.S. Cl. 128/200.22; 222/190; 222/567

(57) ABSTRACT

A fluid dispenser head that is adapted to be assembled on a reservoir containing the fluid, so as to dispense the fluid selectively by a dispenser member, such as a pump or a valve, that is actuated by the dispenser head. The dispenser head includes a dispenser orifice, the dispenser head including a film that incorporates bactericidal and/or bacteriostatic agents, and the film being arranged in the proximity of the dispenser orifice.

The present invention relates to a fluid dispenser head, and to a fluid dispenser device including such a dispenser head.

In the pharmaceutical field, in order to limit the contamination of a fluid, in particular while it is being dispensed through the dispenser head, it is known to apply bactericidal and/or bacteriostatic agents to certain surfaces into which the fluid comes into contact while it is being dispensed. It has thus been proposed to provide such bactericidal and/or bacteriostatic agents to all or part of the expulsion path of the dose, from the inlet of the dip tube, along its passage through the pump, and into the dispenser head, in particular as far as its dispenser orifice through which the fluid is dispensed. Certain devices also include shutters that are arranged inside the dispenser head directly upstream from the dispenser orifice. Nevertheless, each time such a device is used, a residual drop may remain downstream from the dispenser orifice, and in particular in the small recess formed around the orifice by the walls of the dispenser head. The residual drop is particularly likely to become contaminated and next time the device is used, it is thus particularly likely to lead to a risk of the next dose being contaminated. In addition, since a nasal dispenser head is inserted in the user's nose while administering its spray, a large portion of the surface of said head may enter into contact with the walls of the nose and may thus be contaminated. Numerous attempts have been made in order to impart bacteriostatic or bactericidal properties to the plastics materials used to manufacture dispenser heads. Among such solutions, it has been proposed to mold plastics materials that include such bacteriostatic agents, e.g. silver salts or similar, embedded in the plastics material, or to treat the surfaces after molding, in particular by coating the surfaces with layers containing bactericidal agents. Unfortunately, so far, no solution has turned out to be truly satisfactory. Documents FR 2 736 285 and U.S. Pat. No. 6,264,936 describe prior-art devices.

An object of the present invention is to provide a dispenser head and a fluid dispenser device including such a head that do not have the above-mentioned drawbacks.

More particularly, an object of the present invention is to provide a dispenser head that provides effective decontamination of the residual drop that is likely to remain at the outlet of the dispenser orifice after each actuation.

Another object of the present invention is to provide a nasal dispenser head that provides effective decontamination of the surfaces that are likely to become contaminated while it is inserted in the user's nose.

Another object of the present invention is to provide such a dispenser head that does not require modification of the plastics material conventionally used to make dispenser heads.

Another object of the present invention is to provide such a dispenser head that is simple and inexpensive to manufacture and to assemble.

The present invention thus provides a fluid dispenser head that is adapted to be assembled on a reservoir containing said fluid, so as to dispense said fluid selectively by means of a dispenser member, such as a pump or a valve, that is actuated by said dispenser head, said dispenser head including a dispenser orifice, said dispenser head including a film that incorporates bactericidal and/or bacteriostatic agents, said film being arranged in the proximity of said dispenser orifice.

Advantageously, said film is an extruded sheet.

Advantageously, said film has a thickness that is less than 1 millimeter (mm), advantageously less than 0.5 mm.

Advantageously, said dispenser head is a nasal dispenser head for inserting into a user's nostril.

Advantageously, said film extends over the outside of said dispenser head, at least over the portion that penetrates into the nostril.

Advantageously, said dispenser head is molded on said film.

Advantageously, said film is fastened, in particular hot deposited, shrink-fitted, heat-sealed, and/or adhesively-bonded, to said dispenser head, outside and/or inside said dispenser orifice.

Advantageously, said film is in the shape of a disk, having a hole at its center.

Advantageously, said film defines the dispenser orifice.

The present invention also provides a fluid dispenser device comprising: a reservoir containing fluid; and a dispenser member, such as a pump or a valve, mounted on said reservoir; the device further including a dispenser head as described above.

These characteristics and advantages and others of the present invention appear more clearly from the following detailed description, given by way of non-limiting example, and with reference to the accompanying drawings, and in which:

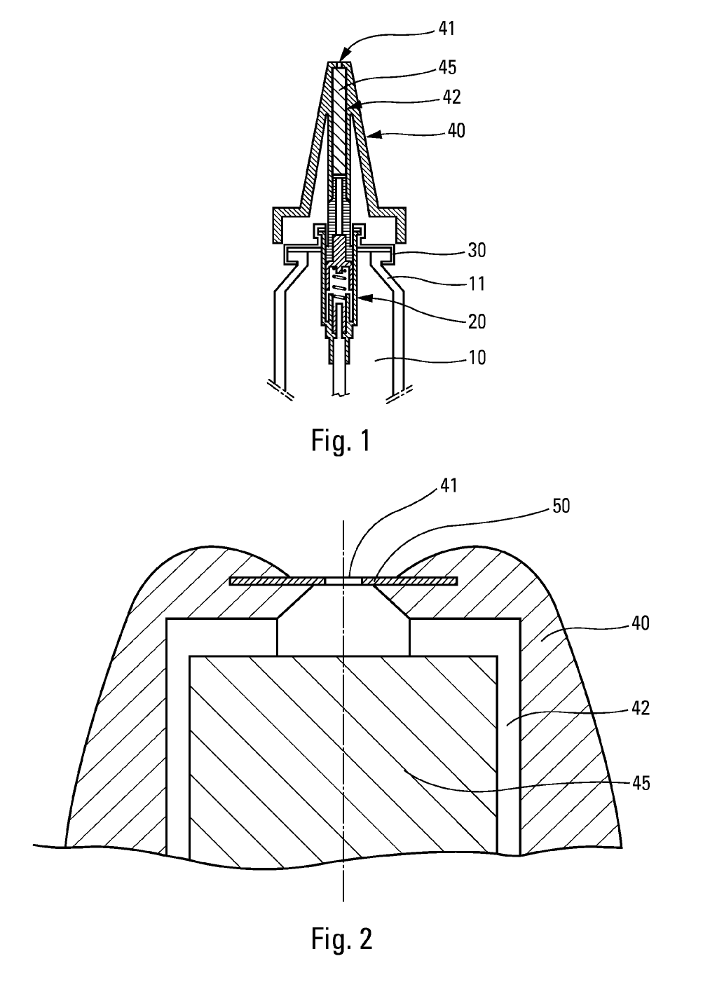

FIG. 1 is a diagrammatic section view of a fluid dispenser device to which the present invention applies;

FIG. 2 is a diagrammatic view of a detail of a portion of the dispenser head in an advantageous embodiment of the present invention;

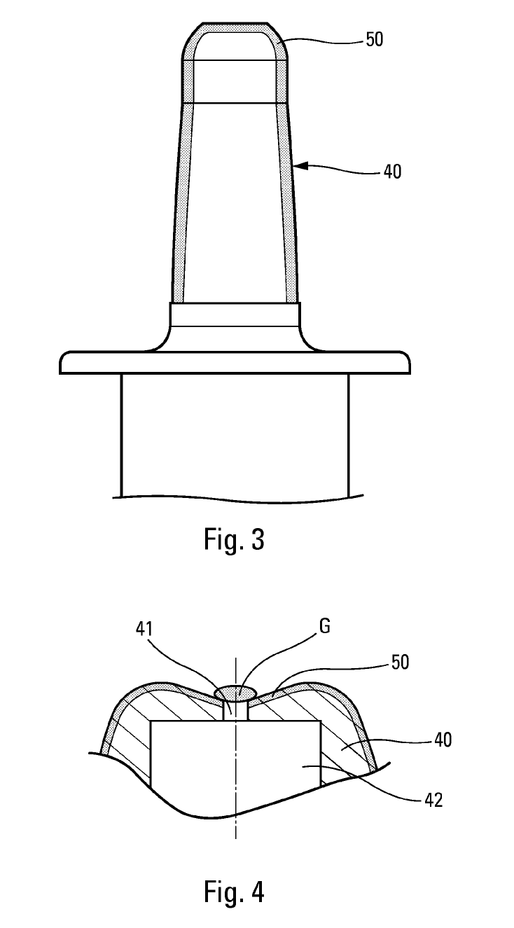

FIG. 3 is a diagrammatic side view of a dispenser head in another embodiment of the invention; and

FIG. 4 is a section view of a detail of FIG. 3.

The techniques that have been used for many years to implement disinfectant treatments of the surfaces that come into contact with the fluid that is to be dispensed, and in particular embedding bactericidal and/or bacteriostatic agents within the plastics material, or coating the surfaces with such agents after they have been molded, have turned out to be unsatisfactory. Research has demonstrated that bactericidal and/or bacteriostatic agents present maximum effectiveness when they are incorporated in a film that is thin, typically about a few tenths of a millimeter thick, and in particular less than 1 mm thick, preferably less than 0.5 mm. Laboratory tests have demonstrated that when disinfectant treatments are performed on thin plastics films of this type, the bactericides and/or bacteriostats turn out to be substantially more effective than when they are applied to thicker surfaces, typically surfaces on a spray head of a fluid dispenser device.

In the present invention, provision is thus made to have a film 50, incorporating bactericidal and/or bacteriostatic agents, at least in the proximity of the dispenser orifice 41 of a dispenser head 40. Such a dispenser head 40 may typically be a nasal dispenser head, as shown diagrammatically in FIGS. 1 and 3, mounted on a pump or a valve 20 that is itself fastened on a reservoir 10 containing the fluid to be dispensed, e.g. by means of a fastener ring 30 that fastens the pump on the neck 11 of the reservoir 10. The dispenser head 40 may typically include an axial dispenser orifice 41 that is provided at the downstream end of an axial extension for penetrating into the user's nostril, an expulsion channel 42 being defined inside said head 40, and an internal nozzle or insert 45 being provided in said expulsion channel 42 so as to limit the amount of dead volume therein and thus provide an expulsion channel of small size that is adapted to ensuring the fluid is sprayed properly through the dispenser orifice 41. A spray profile (not shown) may conventionally be provided at the end of the dispenser head 40, directly upstream from the dispenser orifice 41.

FIG. 2 shows a variant embodiment of the invention in which a film 50 incorporating bactericidal and/or bacteriostatic agents is arranged at the dispenser orifice 41. In the embodiment in FIG. 2, the dispenser head 40 is molded around said film 50 that is made in the form of an extruded disk-shaped sheet having a hole at its center. In this variant embodiment, it is the film 50 itself that defines the dispenser orifice 41 proper. Thus, the residual drop G that is likely to stagnate outside said dispenser orifice 41 is in contact with said film 50, such that the bactericidal and/or bacteriostatic agents contained in said film 50 act effectively on said residual drop, thereby limiting contamination of the next dose. Naturally, the variant embodiment shown in FIG. 2 is only one example, and other variants may be envisaged. For example, the disk could be formed by a rigid plate that incorporates the bactericidal and/or bacteriostatic agents.

FIGS. 3 and 4 show another embodiment of the invention. In this embodiment, the film 50 is applied to the outside of the dispenser head. For a nasal head, as shown in FIG. 3, the film 50 is applied at least to the portion of the head that penetrates into the user's nostril, namely the axial extension that incorporates, at its end, the dispenser orifice 41. Advantageously, the thin film 50 is arranged in an appropriate mold, and the plastics material forming the dispenser head is then injected against said film. In a variant, the thin film may be shaped and/or fastened to the dispenser head by any other means, e.g. hot deposited, shrink-fitted, heat-sealed, and/or adhesively-bonded on the dispenser head, so as to surround the shape of the head. Optionally, the thin film may also extend both inside and outside said dispenser orifice 41. Any other means of applying or fastening such a thin film, incorporating bactericidal and/or bacteriostatic agents, at the dispenser orifice 41 of the dispenser head 40 may also be envisaged. In this embodiment, the important characteristic is that the residual drop G that is likely to remain after each use on the outside of the dispenser orifice 41, is in contact with a thin film that incorporates such bactericidal and/or bacteriostatic agents. In addition, for a nasal dispenser head, any nostril-contact residues that might be present on the outside of the head are also treated.

The invention further presents the advantage of there being no need to modify the plastics materials used for molding the fluid dispenser head. It should not be forgotten that any modification of a material in the context of a fluid dispenser device in the pharmaceutical field is complicated and thus costly, since it must be approved by the various medical and health bodies of various countries. The present invention makes it possible to continue to use the same plastics materials, while guaranteeing effective treatment of the residual drop, as described above. Likewise, the fact of limiting contact between the fluid and a material incorporating bactericidal and/or bacteriostatic agents to the location in which such contact is actually necessary, limits exchanges between the fluid to be dispensed and the bactericidal and/or bacteriostatic agents, as may occur when the treatment is applied to all, or a large portion, of the surfaces that contact said fluid along the dose expulsion path.

Although the present invention is described above with reference to two particular embodiments thereof, the invention is naturally not limited to those embodiments, but, on the contrary, any useful modification could be applied thereto by a person skilled in the art, without going beyond the ambit of the present invention, as defined by the accompanying claims.

1-8. (canceled)

9. A fluid dispenser head that is adapted to be assembled on a reservoir containing said fluid, so as to dispense said fluid selectively by means of a dispenser member, such as a pump or a valve, that is actuated by said dispenser head, said dispenser head including a dispenser orifice, said dispenser head being characterized in that it includes a film that incorporates bactericidal and/or bacteriostatic agents, said film being arranged in the proximity of said dispenser orifice, said dispenser head being a nasal dispenser head for inserting into a user's nostril, said film extending over the outside of said dispenser head, at least over the portion that penetrates into the nostril.

10. A head according to claim 9, wherein said film is an extruded sheet.

11. A head according to claim 9, wherein said film has a thickness that is less than 1 mm, advantageously less than 0.5 mm.

12. A head according to claim 9, wherein said dispenser head is molded on said film.

13. A head according to claim 9, wherein said film is fastened, in particular hot deposited, shrink-fitted, heat-sealed, and/or adhesively-bonded, to said dispenser head, outside and/or inside said dispenser orifice.

14. A head according to claim 9, wherein said film is in the shape of a disk, having a hole at its center.

15. A head according to claim 9, wherein said film defines the dispenser orifice.

16. A fluid dispenser device comprising: a reservoir containing fluid; and a dispenser member, such as a pump or a valve, mounted on said reservoir; the device being characterized in that it further includes a dispenser head according to claim 9.
